(19) United States
(12) Patent Application Publication
(10) Pub. No.: US 2004/0253688 A1
(43) Pub. Date: Dec. 16, 2004

(54) Double selection cloning method ad vectors therefor

(76) Inventors: Fabien Bertaux, Grezieu la Varenne (FR); Florence Lagardette, Saint Etienne Les Oullie (FR); Christine Lapize, Vienne (FR)

Correspondence Address: BURNS DOANE SWECKER & MATHIS L L P, POST OFFICE BOX 1404, ALEXANDRIA, VA, 22313-1404, US

(21) Appl. No.: 10/478,001

(22) PCT Filed: May 17, 2002
(86) PCT No.: PCT/FR02/01682

(30) Foreign Application Priority Data
May 18, 2001 (FR) 01/06568

(51) Int. Cl. C12P 19/34; C12N 15/74

(52) U.S. Cl. 435/91.2; 435/471

(57) ABSTRACT

The invention concerns a novel method for cloning a DNA fragment in a vector, by visual selection using two antibiotics as well as the vectors used for implementing said method, and a kit comprising said vectors.

This invention relates to a new method for cloning a DNA fragment in a vector by a visual selection using two antibiotics, and to vectors that can be used to implement this method, and a kit comprising these vectors.

Cloning of DNA in a vector consists of inserting one or several DNA fragments, possibly but not necessarily in sequence, into a vector in order to build a new vector. It is possible that new problems can arise during these construction steps, including a low efficiency of ligation reactions when the DNA fragment. is introduced, instability of the vector into which the DNA fragment was integrated inducing over-representation of the initial vector, etc.

These difficulties may be observed for any type of vector such as plasmids, cosmids, artificial chromosomes (bacteria, BAC, yeast, YAC or MAC mammals), and particularly for complex constructions such as the construction of homologous recombination vectors for which an attempt is made to introduce large fragments (usually between 2 and 30 kilobases (kb), and preferably between 2 and 7 kb) into appropriate vectors. It may be important to integrate two different DNA fragments consisting of the 3′ and 5′ regions of the locus targeted by recombination, for the homologous recombination.

In particular, it would also be preferable for these vectors to be selected both in the cloning host (frequently a lower host, prokaryote or unicellular eukaryote, the most frequent host being Escherichia coli) and in a target mammal cell (for example an ES cell, preferably murine or derived from a rodent) . In general, the required vectors will have selection genes (particularly antibiotic resistance genes) that are functional in the two organisms.

At the moment, there are some techniques for selecting events to insert a DNA fragment in a vector, particularly the white/blue selection, the DNA fragment being inserted in the lacZ gene and causing extinction of the activity of the said gene (Ullman et al, J. Mol. Biol. 1967, 24, 339-43). However, the background noise for inserts that are difficult to clone remains high, even if positive events can easily be observed by eye.

Elimination of the ccdB activity (Bernard et al, Gene, 1994, 148, 71-4) can also be used. However, preparation of the plasmid before cloning requires the use of another E. coli strain (gyrA462), different from the strain in which the selection is made after cloning.

The two methods described above cannot be used to determine whether or not the DNA fragment was inserted in the required orientation.

This invention relates to a method for cloning (insertion) of a DNA fragment in a vector, the said method eliminating an important proportion of events that are not the result of the desired insertion reaction. Note that with the method according to the invention, it is easy to select DNA cloning events in the required orientation.

Thus, the purpose of this invention is a method for cloning a DNA fragment in a vector A, the said vector A comprising a functional antibiotic I resistance gene in cloning host cells I, and a functional promoter P in the cloning host cells I, comprising steps consisting of:

a) integrating the DNA into the polylinker site of a vector B useable in the cloning host cells II, the said polylinker site being located in a cassette located between two identical or different restriction sites, the said cassette comprising a gene III providing resistance to an antibiotic III in the cloning host cells I, the said gene III not being under the control of a promoter enabling it to be active in the cloning host cells I, the said vector B having a gene II active in the cloning host cells II with resistance to an antibiotic II,

b) excising the said cassette in vector B by cutting with restriction enzymes corresponding to the said restriction sites,

c) making a ligation of the said excised cassette in the said vector A linearized such that the said cassette is inserted under the control of the said functional promoter I in the cloning host cells I,

d) selecting the ligation events at cloning host cells I resistant both to the antibiotic I and the antibiotic III.

Vectors A and B are preferably plasmids, but these vectors may also be cosmids or artificial chromosomes.

The “polylinker” site is a site that enables integration of external DNA and usually comprises restriction sites (between 1-2 and 5-7).

It is interesting to note that since the selection is made by double resistance to antibiotics I and III, it is also possible to obtain oriented cloning to the extent that if the gene III is to be functional, it must be oriented so that it is controlled by the promoter P.

In one particular embodiment of the invention, the cloning host cells I, and the cloning host cells II are prokaryote cells, and preferably Escherichia coli. However, these cells may be eukaryote cells, and cells I and II are not necessarily identical. Therefore, it is possible that cells II are yeast and that cells I are bacteria. Other cloning hosts could be used, for example phages.

In one preferred embodiment of the invention, the said gene III also provides resistance to an antibiotic in eukaryote cells, particularly mammal cells and preferably ES cells of rodents, mice, pigs, sheep, cattle, rabbits or humans. One gene that is very much preferred is the kanamycin resistance gene in prokaryote cells, which is also a neomycin resistance gene in eukaryote cells. It is interesting that the gene III can be used in both types of cells, particularly to select homologous recombination events in eukaryote cells. Those skilled in the art are familiar with different genes with these properties, and for example zeocin, hygromycin B resistance genes or other genes could be used.

Note that in one special embodiment of the invention, a marker gene could be used instead of an antibiotic resistance gene III, to check the presence of the insert. In particular, the lacZ gene or the GFP coding gene could be used.

In one particular embodiment of the invention, the said genes I and II are identical. Different antibiotic resistance genes could be used, and particularly the gene providing resistance to ampicillin. Those skilled in the art will be familiar with antibiotic resistance genes which are used in cloning vectors, and particular examples include hygromycin B, chloramphenicol, tetracycline, and zeocin resistance genes, although this list is not exhaustive.

In one particular embodiment of the invention, the said restriction sites on the side of the cassette containing the polylinker in vector B are rare.

A “rare restriction site” means a restriction site with a cutoff frequency of more than 10 kb, preferably 15 kb, and even better 20 kb in the human or murine genome, in the target organism in general. Rare enzymes include particularly PmeI, SgrAI, RsrII, ClaI, NotI, AscI, PacI, SrfI, .NheI, FseI, NsiI, SceI. “Homing endonucleases” sites should also be mentioned. These enzymes are proteins coded by genes possessing self-splicing introns. These enzymes make site-specific cutoffs in the double strand DNA and in general recognise sites with 18-20 bases or more. In particular, note I-Ppoi, I-CreI, I-CeuI, PI-PsI, I-SceI, PI-SceI. These enzymes are said to be “very rare”.

In one particular embodiment of the invention, the said vector A also possesses a polylinker site to insert a DNA fragment. This has the effect of making it easier to clone the region 5′ or the region 3′ of the required target locus for homologous recombination, since the region 3′ or the region 5′ is preferably integrated in the plasmid A, before the method according to the invention is used with the other region and plasmid B. It is then useful if A is linearized using a rare or very rare restriction enzyme as defined above.

Those skilled in the art will know how to define active promoters P in cloning hosts I. In the preferred embodiments of the invention, the said promoter P is chosen from among the promoter of the transposon Tn5 kanamycin resistance gene, the promoter of the bla gene (ampicillin resistance gene), the promoter of the tryptophan operon Trp, the promoter of the lactose operon, or any other promoter accessible in the PromEC database (hpttp://bioinfo.md.huji.ac.il/marg/promec).

In one special embodiment of the invention, the vectors according to the invention also have the following characteristics:

the said gene III in the said cassette in vector B is under the control of a promoter Eb active in eukaryote cells, particularly mammal cells, or

the said vector A has a promoter Ea active in eukaryote cells, particularly mammal cells, located such that the said gene III is under the control of the said promoter Ea after insertion in vector A.

Those skilled in the art will know which promoters can be used in cloning hosts II. In preferred embodiments for which the hosts II are eukaryote cells, the said promoters Ea and Eb are chosen among promoters of the chicken beta-actin, PGK, thymidine kinase of the herpes simplex virus, SV40, or the “immediate early enhancer” of the human cytomegalovirus.

This embodiment is preferred particularly when the gene III can induce resistance to an antibiotic both in the prokaryote cells and in eukaryote cells, and particularly mammal cells. Coupled hybrid prokaryote-eukaryote promoters controlling the said gene III after use of the method are similar to promoters described particularly for pEGFP-C1 plasmids in Cormack et al (1996, Gene, 173, 33-8) or pGN plasmids in LeMouellic et al (1990, Proc. Natl. Acad. Sci. USA, 87, 4712-6).

As already mentioned, it is often interesting to use the method according to the invention for preparation of vectors intended for homologous recombination in pluricell organism stem cells. Thus, a first fragment (for example 3′ or 5′ of a target locus) is inserted in vector A, the other fragment in inserted in vector B, and the final large vector is constructed using the method according to the invention.

Thus, in one particular case, DNA fragments introduced into the said vector B and optionally into the said vector A are genomic DNA fragments, preferably originating from the same host. In one particularly preferred case, the said fragments are 3′ and 5′ fragments of the same locus, which is a target for homologous recombination.

The host in question is preferably a mammal, but it may also be a yeast, fungus or bacteria. Mammals may include rodents (particularly mice, rats, rabbits), and also pigs, sheep, cattle, dogs, cats and possibly humans.

Therefore, depending on the embodiment, the method according to the invention enables:

a positive visual selection of clones incorporating the desired insert, since these are the only clones that can survive in the selection medium,

time saving on construction of the final vector since the step involving the creation of a large vector is the step in which the positive selection is made. Furthermore, two inserts (for example 5′ and 3′ of the same locus) are inserted in two different vectors in the first step, and not one after the other,

use of the resistance gene III, when it is functional in prokaryote and eukaryote cells, can make the homologous recombination reaction in the eukaryote cells directly after cloning (possibly after linearisation of the final vector or excision of the insert) and thus easily select recombination events in these cells.

Vectors according to this invention are also included within the scope of this invention, alone or in combination in a kit.

Therefore, the purpose of the invention is a kit for cloning DNA fragments in a vector A, comprising:

a vector A, based on a usual cloning vector skeleton in cloning host cells I, the said vector comprising a resistance gene to a functional antibiotic I in the said cells I, with the following from 5′ to 3′,:

possibly a polylinker for inserting DNA fragments in the said vector,

a functional promoter P in the cloning host cells I,

possibly a functional promoter Ea in eukaryote cells, particularly mammal cells, located immediately adjacent to the said promoter (at 3′ or 5′), such that a gene placed under the control of the said promoter Ea is also functional in the cloning host cells I through the effect of the promoter P,

a polylinker containing rare restriction sites,

possibly another polylinker enabling introduction of DNA fragments into the said vector,

a vector B based on a skeleton of a usual cloning vector in cloning host cells II, with the following from 5′ to 3′:

a polylinker containing rare restriction sites,

possibly a functional promoter Eb in eukaryote cells, particularly mammal cells,

a gene III providing resistance to an antibiotic III in the cloning host cells I possibly under the control of the promoter Eb,

possibly a polyadenylation sequence,

a polylinker enabling introduction of DNA fragments in the said vector B,

a polylinker containing rare restriction sites,

Vectors A and B are derived from usual vectors used for cloning DNA fragments. Those skilled in the art understand the term “derived”, which in particular means that the final vector globally has the same skeleton (particularly replication origins and stabilizing elements, etc.) as the original vector from which it is derived. It is important to note that this also means that intergenic elements are preferably conserved. Modifications made in the basic vector are therefore restricted and do not modify the vector replication host, or its fundamental properties (number of copies, insert size, stability, etc.). However it will be possible to consider changing selection genes (resistance to antibiotics), provided that this does not modify other properties of the vector.

In the preferred embodiments of the invention, vectors A and B are derived from vectors pUC19, pBR322, pBluescript, or yeast pRs vectors.

In one preferred embodiment, the vector B has the said functional promoter Eb in the eukaryote cells if there is no functional promoter Ea in the eukaryote cells on vector A.

In another embodiment, neither vector A nor vector B has functional promoters in the eukaryote cells.

In one preferred embodiment, the kit according to the invention also contains instructions for using the method according to the invention.

### DESCRIPTION OF FIGURES

FIG. 1: Example of vectors A and B that can be used in the method according to the invention, according to example 1, version C. Vector A induces resistance to antibiotic I, but not to antibiotic III. Vector B induces resistance to antibiotic II, but not resistance to antibiotic III. Polylinker sr: restriction sites); CH: cloning host cells. Polylinkers 1 and 2: to clone exogenic and preferably genomic DNA.

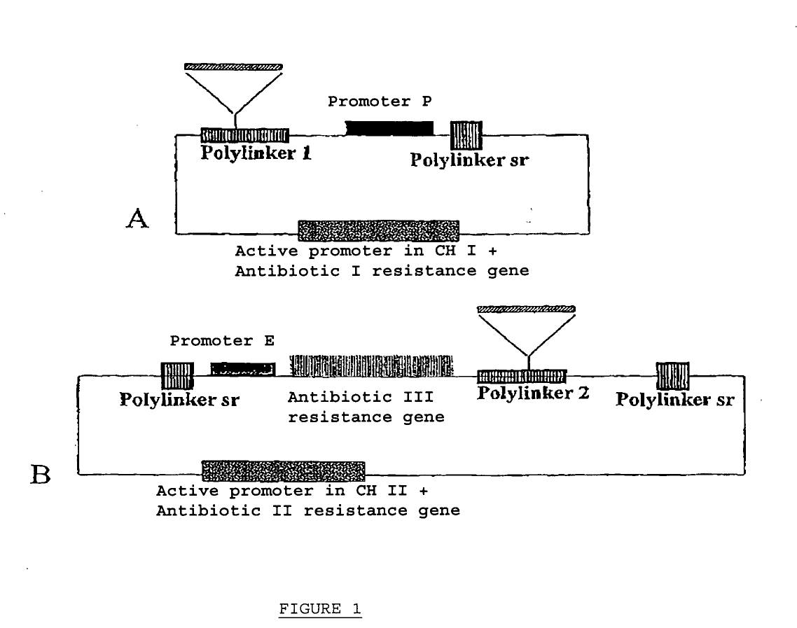

FIG. 2: Final vector obtained after digestion of A and B with a restriction enzyme located in polylinker SR, ligation of the insert derived from B in A, and selection with antibiotics I and III.

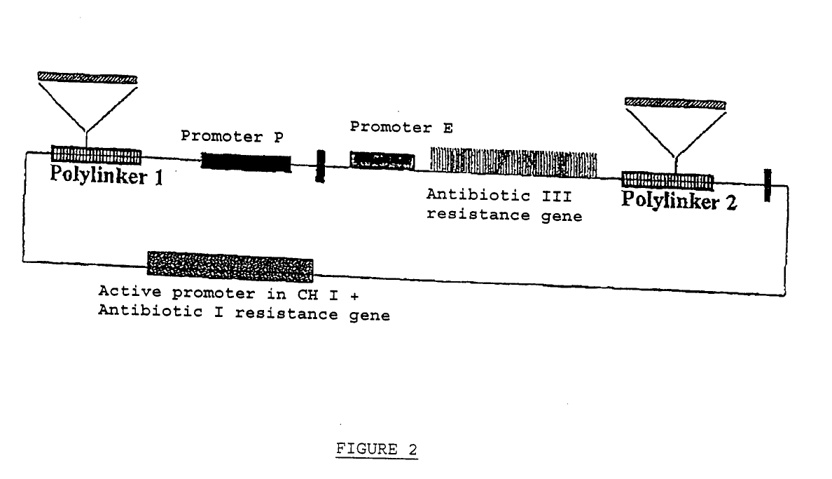

The following example applications are only used to illustrate some aspects of the invention, without restricting it in anyway.

### EXAMPLES

Example 1: construction of plasmids according to the invention

Version A

Plasmid 1: The following are introduced from 5′ to 3′ in a plasmidic skeleton type pUC19 (resistant to ampicillin)

a polylinker for the insertion of a genomic DNA fragment

a prokaryote promoter

a eukaryote promoter coupled to the prokaryote promoter

a polylinker containing rare restriction sites for inserting the insert originating from plasmid 2.

Plasmid 2: The following are introduced from 5′ to 3′ in a plasmidic skeleton type pUC19 (resistant to ampicillin)

a polylinker containing rare restriction sites enabling excision of the insert

cDNA coding for neomycin transferase or any other resistance gene active in prokaryotes and eukaryotes

a polyadenylation sequence

a polylinker enabling introduction of a genomic DNA fragment

a polylinker containing rare restriction sites enabling excision of the insert.

Version B

Plasmid 1: The following are introduced from 5′ to 3′ in a plasmidic skeleton type pUC19 (resistant to ampicillin):

a polylinker for the insertion of a genomic DNA fragment

a eukaryote promoter

a prokaryote promoter coupled to the eukaryote promoter

a polylinker containing rare restriction sites for inserting the insert originating from plasmid 2.

Plasmid 2: The following are introduced from 5′ to 3′ in a plasmidic skeleton type pUC19 (resistant to ampicillin):

a polylinker containing rare restriction sites enabling excision of the insert

cDNA coding for neomycin transferase or any other resistance gene active in prokaryotes and eukaryotes

a polyadenylation sequence

a polylinker enabling introduction of a genomic DNA fragment

a polylinker containing rare restriction sites enabling excision of the insert.

Version C:

Plasmid 1: The following are introduced from 5′ to 3′ in a plasmidic skeleton type pUC19 (resistant to ampicillin):

a polylinker for the insertion of a genomic DNA fragment

a prokaryote promoter

a polylinker containing rare restriction sites for inserting the insert originating from plasmid 2.

Plasmid 2: The following are introduced from 5′ to 3′ in a plasmidic skeleton type pUC19 (resistant to ampicillin):

a polylinker containing rare restriction sites enabling excision of the insert

a eukaryote promoter

cDNA coding for neomycin transferase or any other resistance gene active in prokaryotes and eukaryotes

a polyadenylation sequence

a polylinker enabling introduction of a genomic DNA fragment

Note that in one variant of the method according to the invention, it would be possible to introduce a coding gene for another antibiotic (for example hygromycin) or a marker gene such as the lacZ gene or the GFP gene, instead of the coding gene for neomycin transferase.

1. Method for cloning a DNA fragment in a vector A comprising a functional antibiotic I resistance gene in cloning host cells I, and a functional promoter P in the cloning host cells I, comprising steps consisting of:
a) integrating the DNA into the polylinker site of a vector B useable in the cloning host cells II, the said polylinker site being located in a cassette located between two identical or different restriction sites, the said cassette comprising a gene III providing resistance to an antibiotic III in the cloning host cells I, the said gene III not being under the control of a promoter enabling it to be active in the cloning host cells I, the said vector B having a gene II active in the cloning host cells II with resistance to an antibiotic II,
b) excising the said cassette in vector B by cutting with restriction enzymes corresponding to the said restriction sites,
c) making a ligation of the said excised cassette in the said vector A linearized such that the said cassette is inserted under the control of the said functional promoter I in the cloning host cells I,
d) selecting the ligation events at cloning host cells I resistant both to the antibiotic I and the antibiotic III.

2. Method according to claim 1, characterised in that the cloning host cells I, and the cloning host cells II are prokaryote cells.

3. Method according to claim 1 or 2, characterised in that the said gene III also provides resistance to an antibiotic in eukaryote cells, particularly mammal cells.

4. Method according to any one of claims 1 to 3, characterised in that the said genes I and II are identical.

5. Method according to any one of claims 1 to 4, characterised in that the said restriction sites are rare.

6. Method according to any one of claims 1 to 5, characterised in that the said vector A also possesses a polylinker site which enables or has enabled insertion of a DNA fragment.

7. Method according to any one of claims 2 to 6, characterised in that the said promoter P is chosen from among the promoter of the transposon Tn5 kanamycin resistance gene, the promoter of the bla gene (ampicillin resistance gene) , the promoter of the tryptophan operon Trp, the promoter of the lactose operon, or any other promoter accessible in the PromEC database.

8. Method according to either of claims 1 to 7, characterised in that:
the said gene III in the said cassette in vector B is under the control of a promoter Eb active in eukaryote cells, particularly mammal cells, or
the said vector A has a promoter Ea active in eukaryote cells, particularly mammal cells, located such that the said gene III is under the control of the said promoter Ea after insertion in vector A.

9. Method according to claim 8, characterised in that the said promoter E is chosen among promoters of the chicken beta-actin, PGK, thymidine kinase of the herpes simplex virus, SV40, or the “immediate early enhancer” of the human cytomegalovirus.

10. Method according to either of claims 1 to 8, characterised in that the DNA fragments introduced into the said vector B and optionally into the said vector A are genomic DNA fragments originating from the same host.
